(19) United States
(12) Patent Application Publication
(10) Pub. No.: US 2011/0306564 A1
(43) Pub. Date: Dec. 15, 2011

(54) SOLID COMPOSITION, MICROPARTICLES, MICROPARTICLE DISPERSION LIQUID, AND MANUFACTURING METHODS FOR THESE

(75) Inventors: Gen Takebe, Hamamatsu-shi (JP); Mitsuo Hiramatsu, Hamamatsu-shi (JP); Tokio Takagi, Hamamatsu-shi (JP)

(73) Assignee: HAMAMATSU PHOTONICS K.K.

(21) Appl. No.: 13/217,687

(22) Filed: Aug. 25, 2011

Related U.S. Application Data
(62) Division of application No. 12/235,825, filed on Sep. 23, 2008.

(30) Foreign Application Priority Data
Oct. 2, 2007 (JP) P2007-259094
Oct. 2, 2007 (JP) P2007-259102

(51) Int. Cl. A61K 31/337; A61P 37/06; A61P 35/00; A61P 17/00; A61K 38/13; A61K 31/573

(52) U.S. Cl. 514/20.5; 514/181; 514/449

(57) ABSTRACT

In a dissolving step, in a container 13, a poorly soluble drug and a dispersion stabilizer (polyvinylpyrrolidone and sodium lauryl sulfate) are dissolved in a volatile organic solvent. In a solid composition forming step following the dissolving step, the organic solvent contained in the solution is removed by evaporation, and by the organic solvent removal, a solid composition 1 is obtained as a residue, and the solid composition 1 becomes fixed on the inner wall of the container 13. In a water injecting step following the solid composition forming step, water 2 is injected into the interior of the container 13. In a microparticle dispersion liquid preparing step following the water injecting step, laser light L emitted from a laser light source 11 is irradiated on the solid composition 1 fixed on the inner wall of the container 13 and optical energy is thereby applied to the solid composition 1.

### BACKGROUND OF THE INVENTION

1. Field of the Invention

The present invention relates to a solid composition, microparticles, a microparticle dispersion liquid, and manufacturing methods for these.

2. Related Background Art

In the recent development of new medical drugs, combinatorial chemistry methods have been adopted in synthesizing candidate compounds. Combinatorial chemistry is the art of adopting combinations to synthesize a wide variety of compounds in a short time at one time. Compounds obtained by this method have a solubility problem in many cases. That is, in many cases, even if a compound is found to have excellent physiological activity in itself, if the compound has a property of being difficult to dissolve in water, development of the compound is abandoned. Even with compounds obtained by extraction from natural products, various organic syntheses are carried out and structural optimization is performed to improve solubility. Some medical drugs already on the market are also low in solubility. With such drugs, a drug absorption amount varies within an individual patient and varies among individuals, and this places a large burden in terms of control of levels in blood, etc., on both a physician using a drug and a patient on whom the drug is used.

Microparticle formulations have received attention as a solution to the above problems. With a microparticle formulation, poorly soluble drug particles that are made no more than a micrometer in size are dispersed in water with stability. By using a microparticle formulation, a drug can be increased in absorption rate and amount in a living body. Reduction in variation of absorption amount within an individual patient and among individuals and increase in effective availability with respect to a dose can also be anticipated.

For improving solubility and absorbability of a poorly soluble drug, a drug formulation method such as nanoparticulation by means of pulverization, solid dispersion, or soluble complex formation is selected. However, drug microparticles have a high cohesive property, so that they cannot be stably dispersed in water without adding a proper dispersant. On the other hand, in solid dispersion, a drug in an amorphous state is present in a polymer-based material, so that the solubility in water can be temporarily increased, however, after a predetermined time, the solubility lowers according to precipitation of drug crystals.

Patent Document 1 discloses an invention relating to a solid composition with improved solubility and absorbability. In the invention disclosed in this Document, a poorly soluble drug, a polymer-based material, and a nonionic surfactant are dissolved in an organic solvent, and the solution is spray-dried to obtain a solid composition. In this invention, by dispersing the obtained solid composition in a liquid, microparticles no more than 1 μm maintaining the amorphous nature are obtained.

[Patent Document 1] International Publication WO96/19239

### SUMMARY OF THE INVENTION

Problem to be Solved by the Invention

Patent Document 1 shows a comparative example 4 using sodium lauryl sulfate as an ionic surfactant. However, as a result of this comparative example 4, it is described that, “when sodium lauryl sulfate as an cationic surfactant was used, formation of microparticles no more than 1 μm was not observed.” Therefore, in claim 1 of Patent Document 1, the surfactant is limited to nonionic surfactants.

The present invention was made to solve the above-described problem, and an object thereof is to provide a solid composition, microparticles and a microparticle dispersion liquid etc., which contain a poorly soluble drug and has improved solubility and absorbability, and manufacturing methods for these.

Means for Solving the Problem

A solid composition manufacturing method of the present invention includes: (1) a dissolving step of dissolving a poorly soluble drug, polyvinylpyrrolidone, and sodium lauryl sulfate in a volatile organic solvent; and (2) a solid composition forming step of obtaining a solid composition as a residue by removing the organic solvent contained in the solution obtained through the dissolving step by evaporation.

A microparticle dispersion liquid manufacturing method of the present invention includes: (3) a water injecting step of immersing a solid composition obtained according to the solid composition manufacturing method in water; and (4) a microparticle dispersion liquid preparing step of forming microparticles from the solid composition immersed in water in the water injecting step by applying energy to the solid composition, and manufacturing a microparticle dispersion liquid containing the microparticles dispersed in water.

One container may be used throughout the entirety of the dissolving step, the solid composition forming step, the water injecting step, and the microparticle dispersion liquid preparing step. A container used in the steps before obtaining the residue and a container used in subsequent steps may be different from each other.

According to the present invention, in the dissolving step, a poorly soluble drug, polyvinylpyrrolidone, and sodium lauryl sulfate are dissolved in a volatile organic solvent. In the subsequent solid composition forming step, the organic solvent contained in the solution obtained through the dissolved step is removed by evaporation, and by the organic solvent removal, a solid composition as a residue is obtained. In a further subsequent water injecting step, the solid composition is immersed in water. Then, in the microparticle dispersion liquid preparing step, energy is applied to the solid composition immersed in water and the solid composition is made into microparticles, and a microparticle dispersion liquid containing the microparticles dispersed in water is manufactured.

In the above-described microparticle dispersion liquid preparing step, it is preferable that optical energy is applied to the solid composition to make the solid composition into microparticles, or vibration energy is applied to the solid composition to make the solid composition into microparticles, or energy is applied to the solid composition by stirring water to make the solid composition into microparticles.

A solid composition of the present invention is constituted of a poorly soluble drug, polyvinylpyrrolidone, and sodium lauryl sulfate being molecular-dispersed. Here, molecular-dispersion means uniform dispersion close to the molecular level. Microparticles of the present invention contain a poorly soluble drug, polyvinylpyrrolidone, and sodium lauryl sulfate. A microparticle dispersion liquid of the present invention is obtained by dispersing the microparticles of the present invention in water. A lyophilized material of the present invention is obtained by lyophilizing the microparticle dispersion liquid of the present invention. An orally administered formulation of the present invention contains the microparticles, the microparticle dispersion liquid, or the lyophilized material of the present invention. An injection formulation of the present invention contains a dispersion liquid obtained by resuspending the microparticles, the microparticle dispersion liquid, or the lyophilized material of the present invention in water. These can be manufactured by using the solid composition manufacturing method or microparticle dispersion liquid manufacturing method of the present invention, and have excellent solubility and absorbability.

The present invention can provide a solid composition, microparticles, and a microparticle dispersion liquid, etc., which contain a poorly soluble drug, polyvinylpyrrolidone, and sodium lauryl sulfate and is improved in solubility and absorbability.

The present invention also provides a means for solving the following problem of the conventional technique.

Paclitaxel which is one type of poorly soluble drug is also called taxol, and is known as an anti-tumor drug which effectively works against breast cancer, non-small-cell lung cancer, stomach cancer, esophageal cancer, head and neck cancer, ovary cancer, and uterine body cancer, etc., and is a compound frequently used in clinical practice. This compound is hardly dissolved in water (approximately 0.5 μg/mL at room temperature), so that a drug formulation device is essential for obtaining an injectable solution. At present, a paclitaxel formulation is sold as a mixed solution of ethanol and Cremophor EL (polyoxyethylene castor oil) in the proportion of 50 to 50, and when it is administered, it is mixed with a 5% glucose injection or normal saline and is drip-infused.

However, the above-described paclitaxel formulation contains a large amount of Cremophor EL as a surfactant, so that there is a risk that a patient who has a previous history of hypersensitivity to it will experience an anaphylactic shock. The paclitaxel formulation has a high ethanol concentration even after it is mixed with an infusion solution, so that there is a risk that a patient who is alcohol-hypersensitive will experience alcohol poisoning.

Published Japanese Translation of PCT Application No. H10-502921 discloses an invention relating to stable oil-in-water emulsion containing taxines (taxol) and a manufacturing method for the same. In the invention disclosed in this Document, paclitaxel is dissolved in an alcohol solution, and to this, an equivalent amount of oil is added and mixed until the solution becomes transparent, and then alcohol is removed by rotary evaporation or evaporation under a nitrogen flow. Then, the paclitaxel oil solution obtained through this removal is dispersed in water by using a surfactant, and accordingly, stable oil-in-water emulsion is formed.

Japanese Patent No. 3656550 discloses an invention relating to a drug composition containing cyclodextrin and taxoid. In the invention disclosed in this Document, paclitaxel and cyclodextrin derivative are dissolved in alcohol, and then alcohol is evaporated, and accordingly, a dried solid is obtained.

In the invention disclosed in Published Japanese Translation of PCT Application No. H10-502921, emulsion is formed by using oil, so that the amount to be used for the surfactant is reduced. However, paclitaxel is also a compound with low solubility to oil, so that there is a high possibility that crystals precipitate from the oil emulsion, and this poses a problem in safety.

In the invention disclosed in Japanese Patent No. 3656550, a cyclodextrin derivative is used as a dispersant which replaces the surfactant. However, the amount of cyclodextrin necessary for dispersion is remarkably large as 25 times to 400 times the amount of paclitaxel. The use of such a large amount of additive poses a problem in safety.

To solve the above-described problems, the present invention provides a paclitaxel solid composition, paclitaxel microparticles, and a paclitaxel microparticle dispersion liquid, etc., improved in stability and safety, and manufacturing methods for these.

A paclitaxel solid composition manufacturing method of the present invention includes: (1) a dissolving step of dissolving paclitaxel and a dispersion stabilizer in a volatile organic solvent; and (2) a solid composition forming step of obtaining a paclitaxel solid composition as a residue by removing the organic solvent contained in the solution obtained through the dissolving step by evaporation. Here, it is preferable that the dispersion stabilizer contains polyvinylpyrrolidone and sodium lauryl sulfate.

A paclitaxel microparticle dispersion liquid manufacturing method of the present invention includes: (3) a water injecting step of immersing a paclitaxel solid composition obtained according to the paclitaxel solid composition manufacturing method of the present invention in water; and (4) a microparticle dispersion liquid preparing step of forming microparticles from the paclitaxel solid composition immersed in water in the water injecting step by applying energy to the paclitaxel solid composition, and manufacturing a microparticle dispersion liquid containing the microparticles dispersed in water.

One container may be used throughout the entirety of the dissolving step, the solid composition forming step, the water injecting step, and the microparticle dispersion liquid preparing step. A container to be used in steps before the residue is obtained and a container to be used in subsequent steps may be different from each other.

According to the present invention, in the dissolving step, paclitaxel and a dispersion stabilizer are dissolved in a volatile organic solvent. In the subsequent solid composition forming step, the organic solvent contained in the solution obtained through the dissolving step is removed by evaporation, and by the organic solvent removal, a paclitaxel composition is obtained as a residue. In the further subsequent water injecting step, the paclitaxel solid composition is immersed in water. Then, in the microparticle dispersion liquid preparing step, energy is applied to the paclitaxel solid composition immersed in water and the paclitaxel solid composition is made into microparticles, and a microparticle dispersion liquid containing the microparticles dispersed in water is manufactured.

In the above-described microparticle dispersion liquid preparing step, it is preferable that optical energy is applied to the paclitaxel solid composition to make the paclitaxel solid composition into microparticles, or vibration energy is applied to the paclitaxel solid composition to make the paclitaxel solid composition into microparticles, or energy is applied to the paclitaxel solid composition by stirring water to make the paclitaxel solid composition into microparticles.

A paclitaxel solid composition of the present invention is constituted of paclitaxel and a dispersion stabilizer being molecular-dispersed. Here, molecular dispersion means uniform dispersion close to the molecular level. Paclitaxel microparticles of the present invention contain paclitaxel and a dispersion stabilizer. A paclitaxel microparticle dispersion liquid of the present invention is obtained by dispersing the paclitaxel microparticles of the present invention in water. A lyophilized material of the present invention is obtained by lyophilizing the paclitaxel microparticle dispersion liquid of the present invention. An orally administered formulation of the present invention contains the paclitaxel microparticles, the paclitaxel microparticle dispersion liquid, or the lyophilized material of the present invention. An injection formulation of the present invention contains a dispersion liquid obtained by redispersing the paclitaxel microparticles, the paclitaxel microparticle dispersion liquid, or the lyophilized material of the present invention in water. These can be manufactured by using the paclitaxel solid composition manufacturing method or paclitaxel microparticle dispersion liquid manufacturing method of the present invention, and have excellent safety and stability.

The present invention can provide a paclitaxel solid composition, paclitaxel microparticles, and a paclitaxel microparticle dispersion liquid, etc., which are improved in stability and safety.

### BRIEF DESCRIPTION OF THE DRAWINGS

FIG. 1 is a diagram showing a configuration of a microparticle dispersion liquid manufacturing apparatus 10 to be used in methods for manufacturing a solid composition and microparticle dispersion liquid of a first embodiment of the present invention;

FIG. 2 is a flowchart describing a solid composition manufacturing method and a microparticle dispersion liquid manufacturing method of the first embodiment;

FIG. 3 is a configuration diagram of a modification example of a container 13 used in the first embodiment;

FIG. 4 is a configuration diagram of another modification example of the container 13 used in the first embodiment;

FIG. 5 is a diagram showing particle size distributions of microparticles contained in dispersion liquids A to C, respectively, obtained in Example 1A;

FIG. 6s are electron micrographs of microparticles contained in the dispersion liquids A to C, respectively, obtained in Example 1A;

FIG. 7 is a diagram showing particle size distributions of microparticles contained in dispersion liquids D to F, respectively, obtained in Example 2A;

FIG. 8s are electron micrographs of microparticles contained in the dispersion liquids D to F, respectively, obtained in Example 2A.

FIG. 9 is an HPLC chart of a microparticle dispersion liquid obtained in Example 1B; and

FIG. 10 is an HPLC chart of a microparticle dispersion liquid obtained in Example 1B.

### DESCRIPTION OF THE PREFERRED EMBODIMENTS

Best modes for carrying out the present invention shall now be described in detail with reference to the drawings. In the description of the drawings, elements that are the same shall be provided with the same symbols and overlapping description shall be omitted.

In the embodiment described hereinafter, it is essential that paclitaxel is used as a poorly soluble drug, or polyvinylpyrrolidone and sodium lauryl sulfate are used as a dispersion stabilizer.

In the embodiment described below, “paclitaxel solid composition,” “paclitaxel microparticles,” and “paclitaxel microparticle dispersion liquid” are also referred to respectively as “solid composition,” “microparticles,” and “microparticle dispersion liquid,” simply.

(First Embodiment)

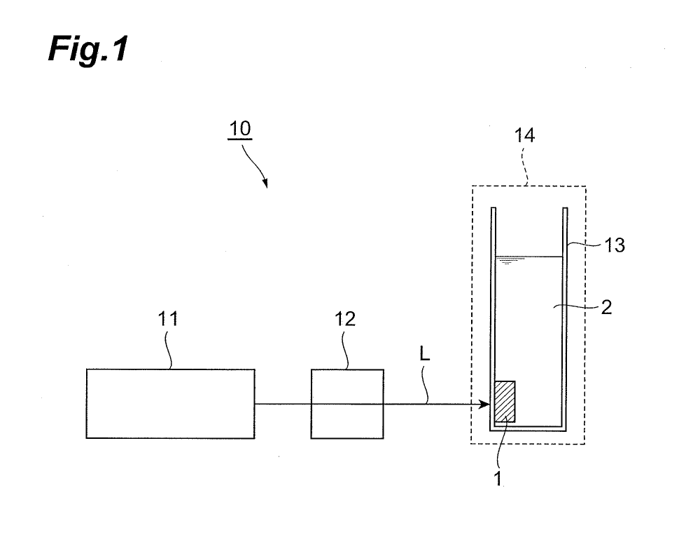

First, a first embodiment of the present invention shall be described. FIG. 1 is a diagram showing a configuration of a microparticle dispersion liquid manufacturing apparatus 10 to be used in manufacturing methods for a solid composition and a microparticle dispersion liquid according to the first embodiment. As shown in this drawing, the microparticle dispersion liquid manufacturing apparatus 10 includes a laser light source 11, an irradiation light controller 12, a container 13, and a temperature controller 14, manufactures a solid composition, constituted of a poorly soluble drug and a dispersion stabilizer being molecular-dispersed, and manufactures a microparticle dispersion liquid in which microparticles containing a poorly soluble drug and a dispersion stabilizer are dispersed in water.

The container 13 is for containing a liquid to be treated, and is composed of a material enabling transmission of a laser light L output from the laser light source 11, and is preferably composed of glass. The temperature controller 14 includes a constant temperature bath, a thermometer, and a temperature control unit, maintains the container 13 housed in the constant temperature bath and the liquid to be treated contained in the interior of the container 13 at a fixed temperature by feedback control by the thermometer and the temperature control unit. A portion of the constant temperature bath, through which the laser light L, output from the laser light source 11, passes, is configured as a transparent window. The laser light source 11 emits a laser light L toward the container 13, and preferably emits an infrared laser light L with a wavelength of no less than 900 nm. The irradiation light controller 12 adjusts both or either of an intensity and an irradiation duration of the laser light L emitted from the laser light source 11 and irradiated on the container 13.

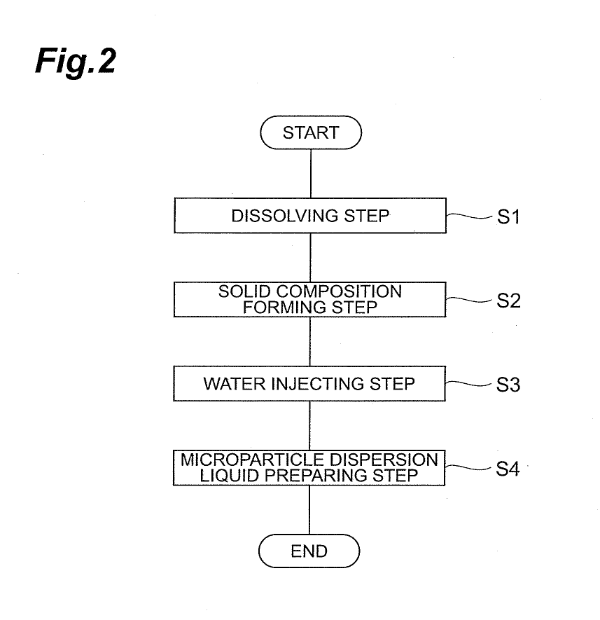

A microparticle dispersion liquid manufacturing method of the first embodiment shall now be described along with a solid composition manufacturing method of the first embodiment. FIG. 2 is a flowchart describing the solid composition manufacturing method and the microparticle dispersion liquid manufacturing method of the first embodiment. With the solid composition manufacturing method of the first embodiment, a solid composition, constituted of a poorly soluble drug and a dispersion stabilizer being molecular-dispersed, is manufactured by successively carrying out a dissolving step S1 and a solid composition forming step S2. With the microparticle dispersion liquid manufacturing method of the first embodiment, a microparticle dispersion liquid in which microparticles containing the poorly soluble drug and a dispersion stabilizer are disposed in water is manufactured by further successively carrying out a water injecting step S3 and a microparticle dispersion liquid preparing step S4 following the dissolving step S1 and the solid composition forming step S2.

In the dissolving step S1, the poorly soluble drug and the dispersion stabilizer are dissolved in a volatile organic solvent in the container 13. Here, the poorly soluble drug is a drug that hardly dissolves in water and although a solubility thereof is not restricted in particular, the solubility is preferably no more than 50 μg/mL at a temperature of 25° C. Commercially available drugs, such as cyclosporin, tacrolimus, nifedipine, nicardipine hydrochloride, phenytoin, digitoxin, diazepam, nitrofurantoin, benoxaprofen, griseofulvin, sulfathiazole, piroxicam, carbamazepine, phenacetin, tolbutamide, theophylline, griseofulvin, chloramphenicol, paclitaxel, camptothecine, cisplatin, daunorubicin, methotrexate, mitomycin C, docetaxel, vincristine, amphotericin B, nystatin, and clobetasone butyrate and other corticosteroids, and other new drug candidate substances under development can be cited as examples of the poorly soluble drug.

The dispersion stabilizer is preferably a polymer or a surfactant. The polymer is preferably a substance that is high in water solubility and is readily soluble in various organic solvents. Hydroxypropylmethylcellulose, methylcellulose, hydroxypropylcellulose, hydroxypropylmethylcellulose phthalate, hydroxypropylmethylcellulose acetate succinate, sodium carboxymethylcellulose, cellulose acetate phthalate, and other cellulose derivatives, agar, gelatin, sodium alginate, polyvinylpyrrolidone, aminoalkylmethacrylate copolymer, methacrylic acid copolymer, carboxyvinyl polymer, polyvinyl alcohol, polyethylene glycol, etc., can be cited as examples of the polymer. The surfactant is preferably of low toxicity, and sodium lauryl sulfate, cholic acid, deoxycholic acid, polyoxyethylene sorbitan fatty acid ester, etc., can be cited as examples.

In particular, polyvinylpyrrolidone and sodium lauryl sulfate are preferably used as the dispersion stabilizer.

As the organic solvent, methanol, ethanol, propanol, and other alcohols, acetone, acetonitrile, methyl acetate, ethyl acetate, diethyl ether, etc., can be cited as examples, and methanol, ethanol, propanol, and other alcohols are more preferable.

In the solid composition forming step S2 following the dissolving step S1, the organic solvent contained in the solution obtained in the dissolving step S1 is removed by evaporation, and by the organic solvent removal, a solid composition 1 as a pellet-form residue is obtained and this solid composition 1 becomes fixed on an inner wall of the container 13. Here, the solid composition 1 obtained is constituted of a poorly soluble drug and a dispersion stabilizer being molecular-dispersed.

In the water injecting step S3 following the solid composition forming step S2, water 2 is injected into the interior of the container 13. By this water injection, the solid composition 1, fixed on the inner surface of the container 13, become immersed in the water 2 (see FIG. 1). Then, in the microparticle dispersion liquid preparing step S4, following the water injecting step S3, the laser light L, emitted from the laser light source 11, is irradiated on the solid composition 1 fixed on the inner surface of the container 13, and optical energy is applied to the solid composition 1. Accordingly, the solid composition 1 is made into microparticles having the most thermally stable structure in water, and a microparticle dispersion liquid, constituted of the microparticles being dispersed in the water 2, is manufactured. This microparticle dispersion liquid is obtained by dispersing the microparticles containing a poorly soluble drug and a dispersion stabilizer in water.

In the present embodiment, because the laser light L is irradiated at high efficiency on the pellet-form solid composition 1 fixed on the inner wall of the container 13, the microparticle dispersion liquid can be manufactured at high efficiency in a short time. Also, because the microparticles are formed even with sufficient weak light irradiation to a degree at which multiphoton absorption processes do not occur, drug degradation and other problems can be suppressed.

Microparticles, containing the poorly soluble drug and the dispersion stabilizer, are manufactured from the microparticle dispersion liquid manufactured as described above. Also, lyophilized microparticles are manufactured by lyophilizing the microparticles. Furthermore, an orally administered formulation, containing the microparticle dispersion liquid, the microparticles, or the lyophilized microparticles, is manufactured, and an injection formulation containing a dispersion liquid obtained by redispersing the microparticle dispersion liquid, the microparticles, or the lyophilized microparticles in water, is manufactured.

Preferably in the microparticle dispersion liquid preparing step S4, the laser light L is irradiated from outside a region of the inner wall of the container 13 on which the solid composition 1 is fixed as shown in FIG. 1 and the irradiated laser light L propagates in the order of the container 13, the solid composition 1, and the water 2. Microparticles are thereby formed near the interface of the solid composition 1 and the water 2 and these microparticles become immediately dispersed in the water 2. Because the laser light irradiation on the interface is constantly performed via the solid composition 1, even when a high concentration of the microparticles is contained in the water 2, the microparticle formation is not lowered in efficiency and the microparticles are formed at a fixed efficiency.

Preferably in the microparticle dispersion liquid preparing step S4, laser light L of a wavelength of no less than 900 nm is irradiated on the solid composition 1 from the laser light source 11. By the laser light L of such a wavelength being irradiated on the solid composition 1, photodegradation of the drug contained in the solid composition 1 can be suppressed further. Also, because the laser light L arrives at the interface via the solid composition 1 and the microparticles are formed at the interface, laser light L of a wavelength of low absorbance with respect to the solid composition 1 is preferably irradiated on the solid composition 1. Specifically, laser light L of a wavelength with which the absorbance with respect to the solid composition 1 is no more than approximately 0.01 is preferably irradiated on the solid composition 1.

Preferably in the microparticle dispersion liquid preparing step S4, both or either of the intensity and the duration of light irradiation on the solid composition 1 are or is adjusted by the irradiation light controller 12, and in this case, it becomes possible to control a particle diameter of the microparticles formed by the light irradiation. Preferably during the light irradiation on the solid composition 1, the irradiated region or the interior of the container is maintained at a fixed temperature by the temperature controller 14, and in this case, the particle diameter of the microparticles formed by the light irradiation is stabilized.

Preferably a sealed container is used as the container 13, and the dissolving step S1, the solid composition forming step S2, the water injecting step S3, and the microparticle dispersion liquid preparing step S4 are performed in a sterilized state. Or, the dissolving step S1 may be performed under a non-sterilized state and after filter sterilization of the solution, the solid composition forming step S2, the water injecting step S3, and the microparticle dispersion liquid preparing step S4 may be performed in a sterilized state. That is, because the present embodiment provides a simple method of simply irradiating light from the exterior of the container 13, it can be put into practice even in a sealed container and an injectable product can also be manufactured readily in a sterilized state.

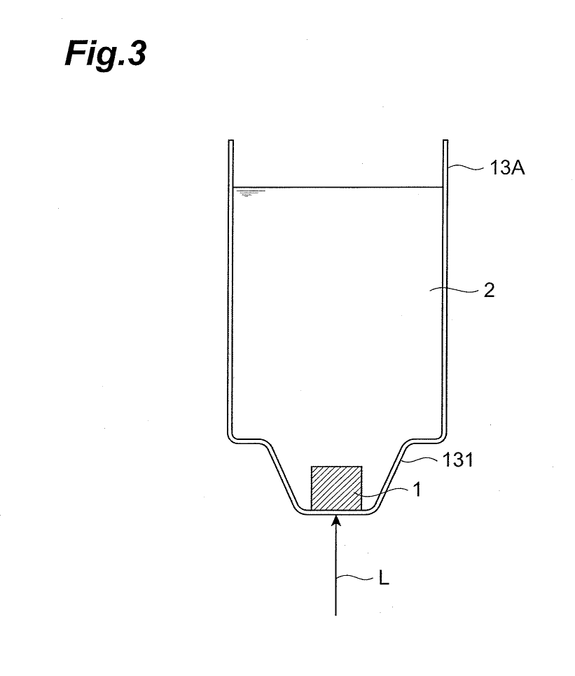

FIG. 3 is a configuration diagram of a modification example of the container 13 to be used in the present embodiment. A container 13A as a modification example of the container 13 shown in this drawing has a hollow 131 for fixing the solid composition 1 on the inner wall. The solid composition 1 can be arranged in the hollow 131 always at a fixed position with respect to the outer wall of the container 13A, and the irradiating position of the laser light L is readily adjusted. Preferably, the hollow 131 is circular as shown in the drawing because it becomes resistant to distortion.

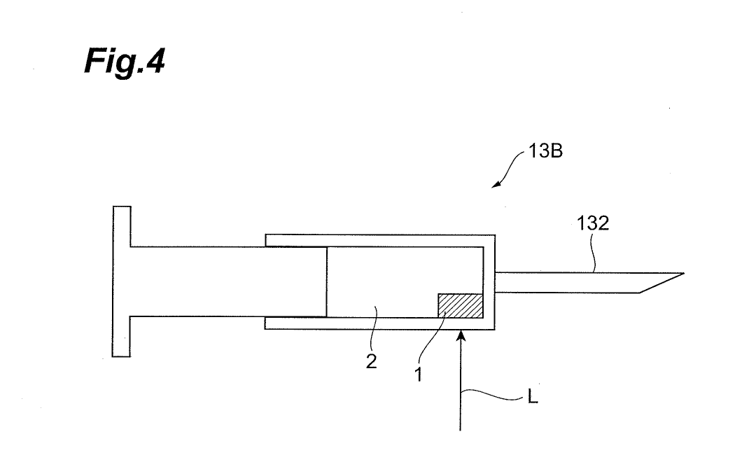

FIG. 4 is a configuration diagram of another modification example of the container 13 to be used in the present embodiment. A container 13B as a modification example of the container 13 shown in this drawing has a function as an injector. The container 13B has an injection needle 132, and has an advantage that the microparticle dispersion liquid immediately after being manufactured can be quickly injected. It is also allowed that, like the container 13A of the above-described modification example, a hollow for fixing the solid composition 1 is provided on the injector inner wall.

The above-described solid composition, microparticles, microparticle dispersion liquid, lyophilized material, oral administrated formulation, and injection formulation when polyvinylpyrrolidone and sodium lauryl sulfate are used as a dispersion stabilizer are respectively excellent in solubility and absorbability as shown in the examples described later.

The above-described paclitaxel solid composition, paclitaxel microparticles, paclitaxel microparticle dispersion liquid, lyophilized material, oral administrated formulation, and injection formulation when paclitaxel is used as a poorly soluble drug are respectively excellent in safety because they contain paclitaxel as a poorly soluble drug and a low-toxic dispersion stabilizer. As shown in the examples described later, these are also excellent in stability.

(Second Embodiment)

A second embodiment of the present invention shall now be described. In comparison with the first embodiment, in the second embodiment, the dissolving step S1, the solid composition forming step S2, and the water injecting step S3 are the same, however, the microparticle dispersion liquid preparing step S4 is different. That is, although optical energy is applied to the solid composition 1 in the microparticle dispersion liquid preparing step S4 in the first embodiment, in the microparticle dispersion liquid preparing step S4 in the second embodiment, vibration (preferably, ultrasonic vibration) energy is applied to the solid composition 1 and the solid composition 1 is pulverized and made into microparticles, and a microparticle dispersion liquid, constituted of the microparticles being dispersed in the water 2, is thereby manufactured.

In the microparticle dispersion liquid preparing step S4 in the second embodiment, an ultrasonic probe as a vibrating unit for applying vibration energy to the solid composition 1 is immersed in the water 2 in the container 13, and ultrasonic vibration generated from this ultrasonic probe is applied to the solid composition 1. Or, as a vibrating unit, an ultrasonic generator is attached to the outer wall of the container 13 and ultrasonic vibration generated from this ultrasonic generator is applied to the solid composition 1. Or, the container 13 is put into an ultrasonic cleaner as a vibrating unit, and ultrasonic vibration is applied to the solid composition 1 from this ultrasonic cleaner. Or, the container 13 is put into a test tube mixer as a vibrating unit, and vibration is applied to the solid composition 1 from this test tube mixer.

In all of these cases, vibration energy is applied to the solid composition 1, and accordingly, the solid composition 1 is made into microparticles having the most thermally stable structure in water, and a microparticle dispersion liquid, constituted of the microparticles being dispersed in the water 2, is thereby manufactured. Thus, in the present embodiment in the case where polyvinylpyrrolidone and sodium lauryl sulfate are used as a dispersion stabilizer, microparticles and a microparticle dispersion liquid, etc., which are improved in solubility and absorbability can also be manufactured.

In the present embodiment in the case where paclitaxel is used as a poorly soluble drug, paclitaxel microparticles and a paclitaxel microparticle dispersion liquid, etc., which are improved in stability and safety can also be manufactured.

(Third Embodiment)

A third embodiment of the present invention shall now be described. In comparison with the first embodiment, in the third embodiment, the dissolving step S1, the solid composition forming step S2, and the water injecting step S3 are the same, however, the microparticle dispersion liquid preparing step S4 is different. That is, although optical energy is applied to the solid composition 1 in the microparticle dispersion liquid preparing step S4 in the first embodiment, in the microparticle dispersion liquid preparing step S4 in the third embodiment, the water 2 in the container 13 is stirred to apply energy to the solid composition 1, and the solid composition 1 is made into microparticles having the most thermally stable structure in water, and a microparticle dispersion liquid, constituted of the microparticles being dispersed in the water 2, is thereby manufactured.

In the microparticle dispersion liquid preparing step S4 in the third embodiment, a magnetic stirrer is used as a stirring unit for stirring the water 2 in the container 13, and by rotation of this magnetic stirrer, the water 2 in the container 13 is stirred. Or, as a stirring unit, a test tube mixer is used, and by vibration of this test tube mixer, the water 2 in the container 13 is stirred. The test tube mixer acts as a vibrating unit when the amount of water 2 in the container 13 is large, however, when the amount of water 2 in the container 13 is small, it acts as a stirring unit.

In all of these cases, energy is applied to the solid composition 1 by stirring the water 2 in the container 13, and the solid composition 1 is made into microparticles having the most thermally stable structure in water, and a microparticle dispersion liquid, constituted of the microparticles being dispersed in the water 2, is thereby manufactured. Thus, in the present embodiment in the case where polyvinylpyrrolidone and sodium lauryl sulfate are used as a dispersion stabilizer, microparticles and a microparticle dispersion liquid, etc., which are improved in solubility and absorbability can also be manufactured. In the present embodiment in the case where paclitaxel is used as a poorly soluble drug, paclitaxel microparticles and a paclitaxel microparticle dispersion liquid, etc., which are improved in stability and safety can also be manufactured.

### EXAMPLE 1A

A detailed Example 1A of a solid composition and a microparticle dispersion liquid of the present embodiment shall now be described. In Example 1A described below, cyclosporin A was used as a poorly soluble drug, and a microparticle dispersion liquid in which microparticles containing cyclosporin A, polyvinylpyrrolidone, and sodium lauryl sulfate were dispersed in water was manufactured. All following operations were carried out under room temperature (20° C.).

Cyclosporin A bulk powder (10 mg) as a poorly soluble drug, and polyvinylpyrrolidone (50 mg) and sodium lauryl sulfate (2 mg) as a dispersion stabilizer were placed in a test tube and dissolved in ethanol (1 mL), which is a volatile organic solvent. Under reduced pressure conditions, ethanol was dried, and a solid composition in which the drug (cyclosporin A) and the dispersion stabilizer (polyvinylpyrrolidone and sodium lauryl sulfate) were uniformly molecular-dispersed was thereby obtained. The solid composition thus obtained was hermetically sealed upon adding water to the test tube.

Nd:YAG pulse laser light was irradiated on the solid composition in the test tube from the lateral side of the test tube. Irradiation conditions were a wavelength of 1064 nm, an irradiation light intensity of 0.61 J/cm²/pulse, a pulse width of 5 to 7 ns, and a repetition frequency of 10 Hz. After 10 minutes of irradiation, a uniformly cloudy dispersion liquid A was obtained upon shaking gently.

Also, ultrasonic vibration was applied to the solid composition in the test tube. The apparatus used was a desktop ultrasonic cleaner B5510 (manufactured by Branson Ultrasonics Corp.), and the treatment conditions were 180 J/sec (42 kHz). After the treatment for 10 minutes, a uniformly cloudy dispersion liquid B was obtained.

Furthermore, the solid composition in the test tube was vibrated and stirred. The apparatus used was a test tube mixer HM-10H (manufactured by As One Corp.). After the treatment for 10 minutes, a uniformly cloudy dispersion liquid C was obtained.

A cyclosporin A amount contained in the obtained microparticle dispersion liquid was quantified by measuring an absorbance at a wavelength of210 nm by using high performance liquid chromatography (hereinafter referred to as “HPLC”). All the dispersion liquids A to C obtained according to each of the three energy applying methods showed a concentration of 9 to 10 mg/mL in the HPLC quantification.

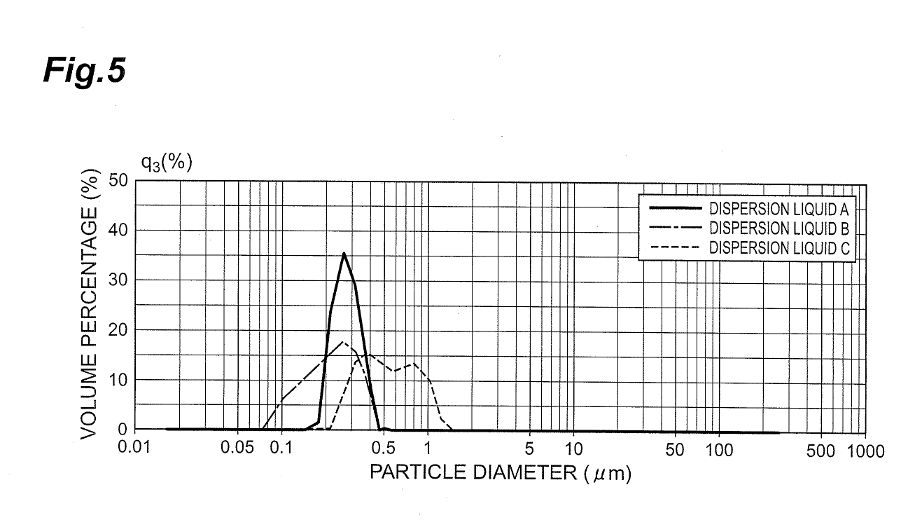

Particle diameter distributions of these dispersion liquids A to C were measured. FIG. 5 is a diagram showing particle size distributions of microparticles contained in the dispersion liquids A to C, respectively, obtained in Example 1A. SALD-7000 (manufactured by Shimadzu Corp.) was used as a measuring apparatus for particle diameter measurement. All of the dispersion liquids A to C are considered to be uniform microparticle dispersion liquids of a uniform particle size. The dispersion liquid A obtained by laser light irradiation, having a particle size distribution range of 50 to 450 nm, had a particle diameter with a peak of 250 nm.

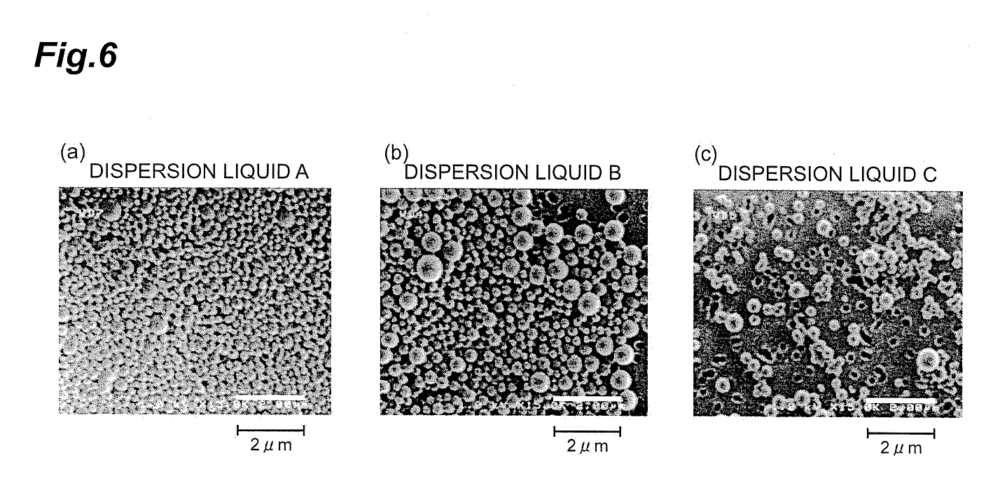

FIGS. 6 are electron micrographs of the microparticles contained in the dispersion liquids A to C, respectively, obtained in Example 1A. A scanning electron microscope S4200 (manufactured by Hitachi, Ltd.) was used as a measuring apparatus. As can be observed from the micrographs, the microparticles have a spherical shape in all dispersion liquids A to C, and this matches the particle size distribution data of FIG. 5 and the microparticles are thus considered as being a uniform assembly of microparticles. Numerous microparticles with a particle diameter of approximately 200 to 300 nm were observed in the dispersion liquid A obtained by laser light irradiation.

As described above, it was possible to prepare a microparticle dispersion liquid, in which cyclosporin A microparticles were dispersed according to each of the three energy applying methods of laser light irradiation, vibration (including ultrasonic vibration), and stirring. Sedimentation was hardly observed even when all the dispersion liquids A to C obtained were left to stand still at room temperature for several days. Furthermore, lyophilization of these microparticle dispersion liquids A to C was possible, and significant differences in the electron microscopy image were not observed between the state before lyophilization and a resuspended dispersion liquid.

### EXAMPLE 2A

A more detailed example 2A of the solid composition and the microparticle dispersion liquid of the present embodiment will now be described. In the present Example 2A described hereinafter, clobetasone butyrate was used as the poorly soluble drug and a microparticle dispersion liquid in which microparticles containing clobetasone butyrate, polyvinylpyrrolidone, and sodium lauryl sulfate were dispersed in water, was manufactured. All following operations were carried out under room temperature (20° C.).

Clobetasone butyrate bulk powder (10 mg) as the poorly soluble drug and polyvinylpyrrolidone (50 mg) and sodium lauryl sulfate (2 mg) as the dispersion stabilizer were placed in a test tube and dissolved in ethanol (1 mL), which is a volatile organic solvent. The ethanol was dried under reduced pressure conditions to obtain a solid component in which the drug (clobetasone butyrate) and the dispersion stabilizer (polyvinylpyrrolidone and sodium lauryl sulfate) were molecular-dispersed uniformly. The solid composition thus obtained was hermetically sealed upon adding water to the test tube.

Nd:YAG pulse laser light was irradiated on the solid composition in the test tube from the lateral side of the test tube.

Irradiation conditions were a wavelength of 1064 nm, an irradiation light intensity of 0.61 J/cm²/pulse, a pulse width of 5 to 7 ns, and a repetition frequency of 10 Hz. After 10 minutes of irradiation, a uniformly cloudy dispersion liquid D was obtained upon shaking gently.

Also, ultrasonic vibration was applied to the solid composition in the test tube. The apparatus used was a desktop ultrasonic cleaner B5510 (manufactured by Branson Ultrasonics Corp.), and the treatment conditions were 180 J/sec (42 kHz). After the treatment for 10 minutes, a uniformly cloudy dispersion liquid E was obtained.

Furthermore, the solid composition in the test tube was vibrated and stirred. The apparatus used was a test tube mixer HM-10H (manufactured by As One Corp.). After the treatment for 10 minutes, a uniformly cloudy dispersion liquid F was obtained.

A clobetasone butyrate amount contained in the obtained microparticle dispersion liquid was quantified by measuring an absorbance at a wavelength of 240 nm by using HPLC. The dispersion liquids D to F obtained according to each of the three energy applying methods showed a concentration of 9 to 10 mg/mL in the HPLC quantification.

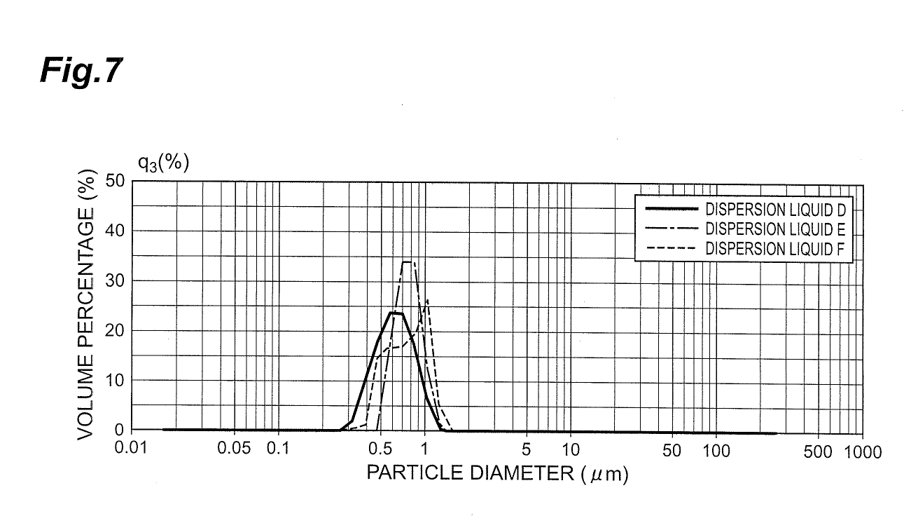

Particle diameter distributions of these dispersion liquids D to F were measured. FIG. 7 is a diagram showing particle size distributions of microparticles contained in each of the dispersion liquids D to F obtained in Example 2A. SALD-7000 (manufactured by Shimadzu Corp.) was used as a measuring apparatus for particle diameter measurement. All of the dispersion liquids D to F are considered to be uniform microparticle dispersion liquids of a uniform particle size. The dispersion liquid D obtained by laser light irradiation, having a particle size distribution range of 300 to 1500 nm, had a particle diameter with a peak of 600 nm.

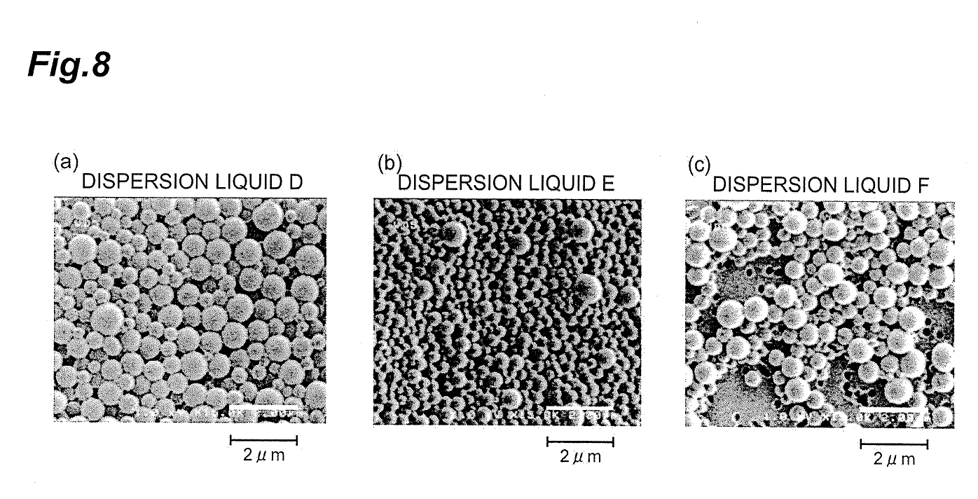

FIGS. 8 are electron micrographs of the microparticles contained in the dispersion liquids D to F, respectively, obtained in Example 2A. A scanning electron microscope S4200 (manufactured by Hitachi, Ltd.) was used as a measuring apparatus. As can be observed from the micrographs, the microparticles have a spherical shape in all the dispersion liquids D to F, and this matches the particle size distribution data of FIG. 7 and the microparticles are thus considered as being a uniform assembly of microparticles. Numerous microparticles with a particle diameter of approximately 500 to 600 nm were observed in the dispersion liquid D obtained by laser light irradiation.

As described above, it was possible to prepare a microparticle dispersion liquid, in which clobetasone butyrate microparticles are dispersed according to each of the three energy applying methods of laser light irradiation, vibration (including ultrasonic vibration), and stirring. Sedimentation was hardly observed even when all the dispersion liquids D to F were left to stand still at room temperature for several days. Lyophilization of all these microparticle dispersion liquids D to F obtained was possible, and significant differences in the electron microscopy image were not observed between the state before lyophilization and a resuspended dispersion liquid.

### EXAMPLE 1B

A more detailed example of the paclitaxel solid composition and the paclitaxel microparticle dispersion liquid shall now be described.

In the present example described below, all following operations were carried out under room temperature (20° C.).

Paclitaxel bulk powder (10 mg) as a poorly soluble drug, and polyvinylpyrrolidone (50 mg) and sodium lauryl sulfate (2 mg) as a dispersion stabilizer were placed in a test tube and dissolved in ethanol (1 mL), which is a volatile organic solvent. Under reduced pressure conditions, ethanol was dried, and a paclitaxel solid composition in which the drug (paclitaxel) and the dispersion stabilizer (polyvinylpyrrolidone and sodium lauryl sulfate) were uniformly molecular-dispersed was thereby obtained. The paclitaxel solid composition thus obtained was hermetically sealed upon adding water to the test tube.

Nd:YAG pulse laser light was irradiated on the paclitaxel solid composition in the test tube from the lateral side of the test tube. Irradiation conditions were a wavelength of 1064 nm, an irradiation light intensity of 0.61 J/cm²/pulse, a pulse width of 5 to 7 ns, and a repetition frequency of 10 Hz. After 10 minutes of irradiation, a substantially colorless transparent paclitaxel microparticle dispersion liquid was obtained upon shaking gently.

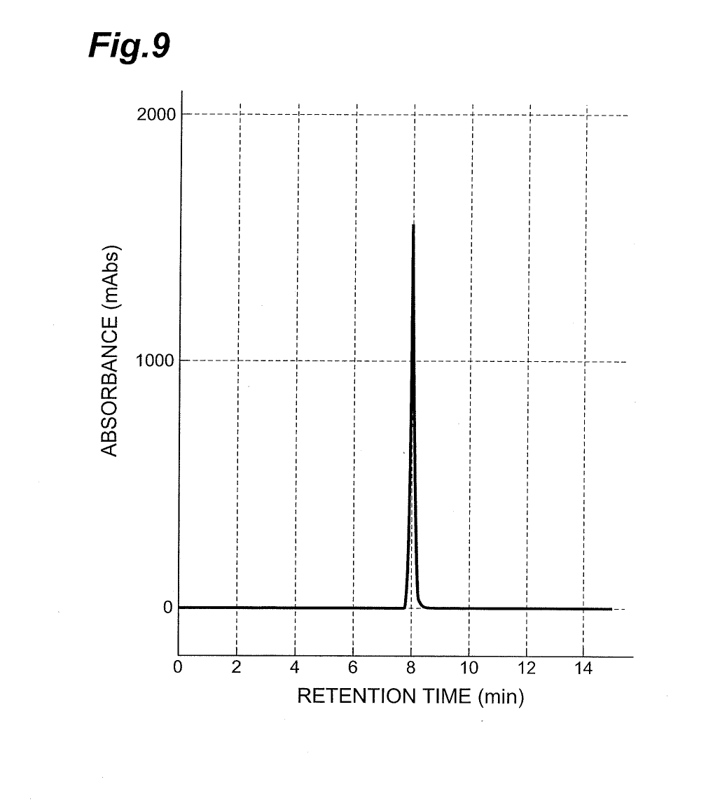

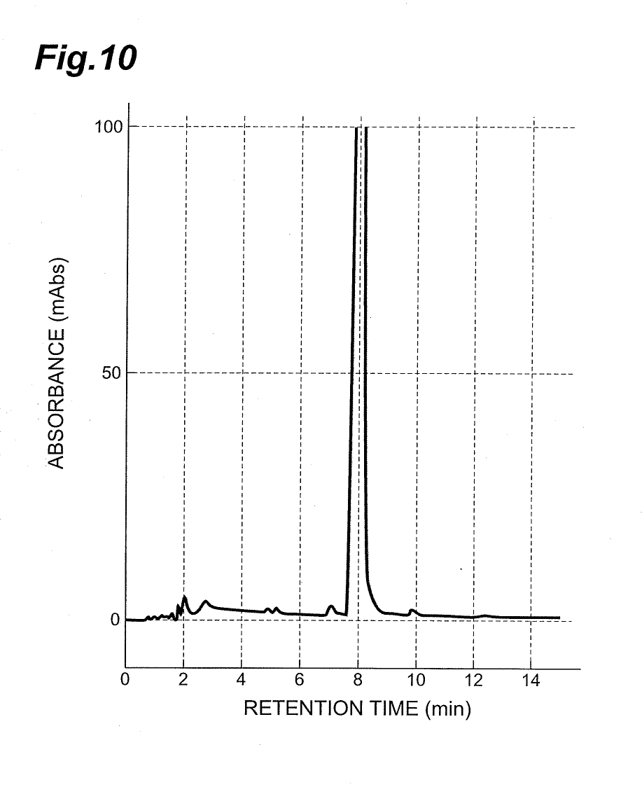

A paclitaxel amount contained in the obtained paclitaxel microparticle dispersion liquid was quantified by measuring an absorbance at a wavelength of 227 nm by using high performance liquid chromatography (hereinafter referred to as “HPLC”). FIG. 9 and FIG. 10 are HPLC charts of the paclitaxel microparticle dispersion liquid obtained in Example 1B. FIG. 10 shows a part of FIG. 9 in an enlarged manner. ODS-C18 (manufactured by Tosoh Corp.) was used as a separation substrate and an acetonitrile-water mixed solution (1:1) was used as a mobile phase to carry out the chromatography at a temperature of 37° C. A solution, prepared by dissolving paclitaxel bulk powder in methanol to a concentration of 0.5 mg/mL, was used as a reference preparation.

As shown in these drawings, paclitaxel was eluted at a position of approximately 8 minutes, and as a result of comparing and calculating the paclitaxel amount in the sample based on the peak area obtained by measuring the reference preparation, the paclitaxel amount in the paclitaxel microparticle dispersion liquid was found to be 9.80 mg/mL (n=3). It was thus possible to prepare a paclitaxel microparticle dispersion liquid with a sufficiently high concentration approximately 20,000 times as high as the solubility (approximately 0.5 μg/mL) of paclitaxel in water. On the HPLC chart, the impurity peak is approximately one several hundredths to one several thousandths of the principal agent peak, and an increase in the impurity peak due to laser light irradiation was not observed.

A particle diameter distribution of the obtained paclitaxel microparticle dispersion liquid was measured by using a measuring apparatus for particle size distribution SALD-7000 (manufactured by Shimadzu Corp.). Furthermore, an electron microscopic image (SEM) of microparticles contained in the paclitaxel microparticle dispersion liquid was observed by using a scanning electron microscope S4200 (manufactured by Hitachi, Ltd.). However, neither of the particle diameter distribution (measurement limit<approximately 100 nm) nor the electron microscopic image (measurement limit<approximately 50 nm) could be observed. The reason for this is considered that the particle diameter is less than the measurement limit.

The obtained paclitaxel microparticle dispersion liquid was high in stability, and even after 12 hours elapsed, still had a stably high concentration of 10 mg/mL and was a still substantially colorless and transparent dispersion liquid, and no precipitate was observed.

1. A solid composition manufacturing method comprising:
a dissolving step of dissolving a poorly soluble drug, polyvinylpyrrolidone, and sodium lauryl sulfate in a volatile organic solvent; and
a solid composition forming step of obtaining a solid composition as a residue by removing the organic solvent contained in the solution obtained through the dissolving step by evaporation.

2. A microparticle dispersion liquid manufacturing method comprising:
a water injecting step of immersing a solid composition obtained according to the solid composition manufacturing method of claim 1 in water; and
a microparticle dispersion liquid preparing step of forming microparticles from the solid composition immersed in water in the water injecting step by applying energy to the solid composition, and manufacturing a microparticle dispersion liquid containing the microparticles dispersed in water.

3. The microparticle dispersion liquid manufacturing method of claim 2, wherein in the microparticle dispersion liquid preparing step, optical energy is applied to the solid composition to make the solid composition into microparticles.

4. The microparticle dispersion liquid manufacturing method of claim 2, wherein in the microparticle dispersion liquid preparing step, vibration energy is applied to the solid composition to make the solid composition into microparticles.

5. The microparticle dispersion liquid manufacturing method according to claim 2, wherein in the microparticle dispersion liquid preparing step, energy is applied to the solid composition by stirring the water to make the solid composition into microparticles.

6-15. (canceled)

16. A paclitaxel solid composition manufacturing method comprising:
a dissolving step of dissolving paclitaxel and a dispersion stabilizer in a volatile organic solvent; and
a solid composition forming step of obtaining a paclitaxel solid composition as a residue by removing the organic solvent contained in the solution obtained through the dissolving step by evaporation.

17. The paclitaxel solid composition manufacturing method of claim 16, wherein the dispersion stabilizer contains polyvinylpyrrolidone and sodium lauryl sulfate.

18. A paclitaxel microparticle dispersion liquid manufacturing method comprising:
a water injecting step of immersing a paclitaxel solid composition obtained according to the paclitaxel solid composition manufacturing method of claim 16 in water; and
a microparticle dispersion liquid preparing step of forming microparticles from the paclitaxel solid composition immersed in water in the water injecting step by applying energy to the paclitaxel solid composition, and manufacturing a microparticle dispersion liquid containing the microparticles dispersed in water.

19. The paclitaxel microparticle dispersion liquid manufacturing method of claim 18, wherein in the microparticle dispersion liquid preparing step, optical energy is applied to the paclitaxel solid composition to make the paclitaxel solid composition into microparticles.

20. The paclitaxel microparticle dispersion liquid manufacturing method according to claim 18, wherein in the microparticle dispersion liquid preparing step, vibration energy is applied to the paclitaxel solid composition to make the paclitaxel solid composition into microparticles.

21. The paclitaxel microparticle dispersion liquid manufacturing method of claim 18, wherein in the microparticle dispersion liquid preparing step, energy is applied to the paclitaxel solid composition by stirring the water to make the paclitaxel solid composition into microparticles.

22-31. (canceled)
